(19) United States
(12) Patent Application Publication
(10) Pub. No.: US 2004/0127813 A1
(43) Pub. Date: Jul. 1, 2004

(54) Dual balloon catheter with sensor for continuous transvenous measurement of intracranial pressure

(76) Inventor: Lee H Schwamm, Newton, MA (US)

Correspondence Address: Jason A Berstein, Powell Goldstein Frazer & Murphy, 16th Floor, 191 Peachtree Street NE, Atlanta, GA, 30303-1736, US

(21) Appl. No.: 10/469,651

(22) PCT Filed: Dec. 21, 2001
(86) PCT No.: PCT/US01/49747

(51) Int. Cl. A61B 5/00

(52) U.S. Cl. 600/561

(57) ABSTRACT

An apparatus for the measurement of intracranial pressure in an area proximate to the jugular bulb from a minimally invasive insertion point exterior to the cranial cavity, comprising a narrow diameter intravascular catheter (20) and a conventional guidewire (32, not shown), a first proximal balloon (22) and a second distal balloon (24), an aperture (26) in the distal portion of the catheter (20) for accommodating a sensor (28) or emitter and an aperture (30) in the distal end (31) for accommodating the guidewire (32). An inflation/deflation mechanism (37) is connected to the catheter via an aperture (34) in the external wall of the catheter (20).

### FIELD OF THE INVENTION

The present invention relates to catheters and associated devices for continuous measurement of intracranial pressure via a transvenous approach.

### BACKGROUND OF THE INVENTION

Typically, increased intracranial pressure (“ICP”) in patients occurs with brain swelling due to large strokes, subarachnoid hemorrhages, traumatic brain injury (“TBI”), brain tumors, neurosurgical procedures and in people with liver failure. Over $30 billion annually is spent on direct medical care costs for patients with stroke or TBI. Stroke is the third leading killer in the United States, and TBI is a leading killer of the young. Monitoring of ICP often requires urgent placement of expensive devices by highly skilled physicians (e.g., neurosurgeons) who are often in short supply and not immediately available.

ICP has been monitored by devices that require a craniotomy (a hole drilled in the skull) performed by a neurosurgeon. This may be accomplished with a subarachnoid, subdural or epidural pressure sensor, or intraparenchymal fiber optic pressure sensor or an intraventricular catheter (i.e., ventriculostomy).

Disadvantages of such techniques are the required cutting of a hole in the skull, increased risk for brain hemorrhage, infection, device failure and measurement error (e.g., drift). These devices are associated with some surgical risks and may be contraindicated in patients with baseline abnormal bleeding parameters or those on heparin or other anticoagulants. Patients with raised ICP are at risk for secondary brain injury due to low blood flow to the brain, and many invasive parameters including ICP need to be constantly monitored in these patients.

There is a need for a minimally invasive ICP measurement device that would lower these risks and obviate a craniotomy. It would also be desirable to have such a device that was safer for patients with blood clotting abnormalities. Such a devices would be lower cost and would be more easily calibratable and replaceable. An ideal minimally invasive device could also be used in an outpatient procedure, thus substantially lowering the overall cost to the patient and the healthcare system.

### BRIEF DESCRIPTION OF THE DRAWINGS

The invention is illustrated in the drawings in which like reference characters designate the same or similar parts throughout the figures of which:

FIG. 1 is a side schematic view of a dual balloon catheter according to a preferred embodiment of the present invention.

FIG. 2 is a side cutaway view of a blood vessel with the dual balloon catheter of FIG. 1 positioned therein and the balloons deployed.

FIG. 3 is a side cutaway view and schematic diagram of the connections between the catheter and the electromechanical elements of a preferred embodiment of the present invention.

FIG. 4 is a schematic diagram of the system bus and components of the central processing unit.

FIG. 5 is a schematic view of a patient and the catheter of FIG. 1 in position.

FIG. 6 is a side view in partial cutaway of an alternative embodiment of the present invention in which an additional lumen is incorporated having an aperture. between the balloons.

FIG. 7 is a side view of an alternative embodiment in which a flexible probe extends from the tip of the catheter.

FIG. 8 is a schematic view of a patient and the catheter of FIG. 6 containing an emitter and a detector, and also shows an extracranial detector array.

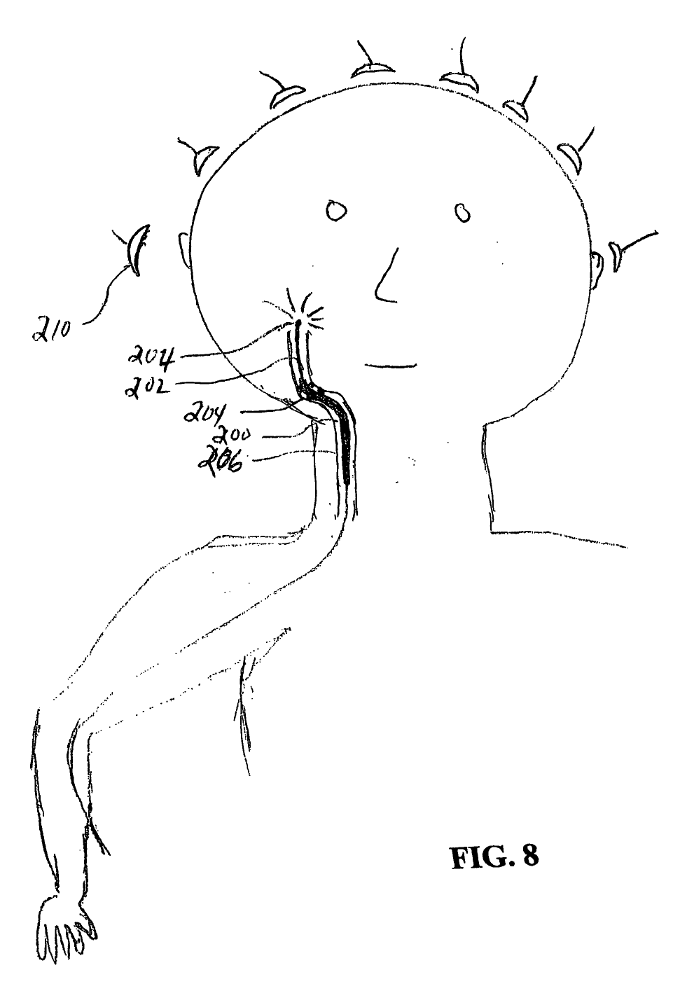

FIG. 9 shows a side cutaway view of an alternative embodiment in which the catheter tip has a lumen extending therefrom capable of introducing a material beyond the tip into the blood vessel.

FIGS. 10A and B are graphs showing flow measurements of ICP and central venous pressure (in mmHg) over time for a patient, showing the correlation between.the two.

### DETAILED DESCRIPTION OF THE PREFERRED EMBODIMENTS

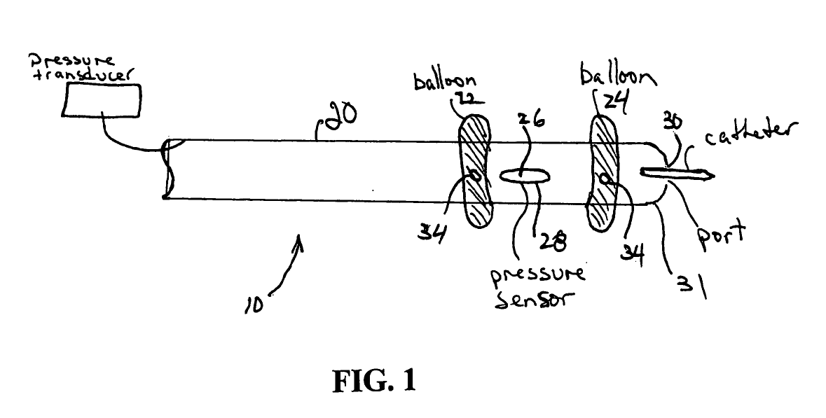

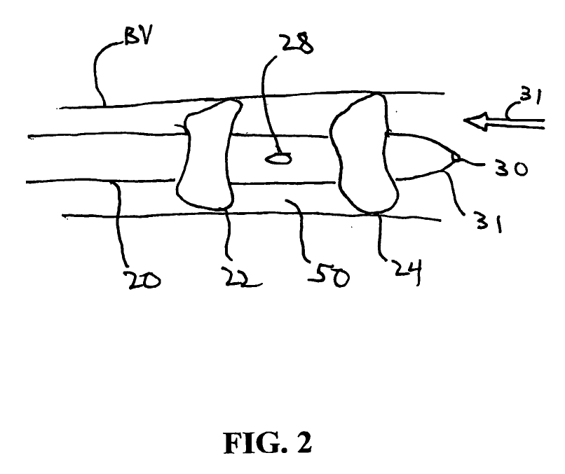

In general, the present invention provides an apparatus for the measurement of ICP in an area proximate to the jugular bulb from a minimally invasive insertion point exterior to the cranial cavity. In a preferred embodiment of the present invention, the apparatus 10 of the present invention comprises a narrow diameter intravascular catheter 20 and a conventional guidewire (not shown, but known to those skilled in the art), a first proximal balloon 22 and a second distal balloon 24, as shown inflated in FIGS. 1 and 2. The catheter 20 is constructed of a biologically insert flexible material as is known to those skilled in the art and can preferably have an external diameter less than or equal to about 7 French or about 2.31 mm for neck insertion of about 8 French (about 2.64 mm) for transfemoral insertion. One of ordinary skill in the art can appreciate that larger or smaller diameters can be used depending on the insertion point or other factors. The distal portion of the catheter 20 has an aperture 26, preferably in the sidewall, for accommodating a sensor 28 or an emitter, as will be described in further detail herein. The catheter 20 also has an aperture 30 at its distal end 31 for accommodating a guidewire 32 (not shown), additional sensor 33 (not shown) or other component. The distal end is preferably rounded to facilitate insertion and advancement in the blood vessel.

The balloons 22 and 24 are constructed of an expandable biologically inert material known to those skilled in the art or developed hereafter. The balloon shape can be conventional torus or other regular or irregular shape. In a preferred embodiment of the present invention, shown in FIG. 2, the balloons 22 and 24 when inflated are slightly angled opposing the direction of flow so that blood flow enhances and maximizes deployment so as to effectively occlude the blood vessel lumen BV. The balloon 22 is attached to the external wall of the catheter 20 by conventional techniques such as, but not limited to, adhesive, sonic welding or the like. An aperture 34 in the catheter 20 external wall is connected to an inflation/deflation mechanism 37 (described in greater detail hereinbelow). Typically, the balloons 22 and 24 are inflated by being in fluid (or gaseous) communication with the inflation mechanism and a gas such as, but not limited to, air, oxygen, nitrogen other biologically inert gas, or a liquid, such as but not limited to, saline, water or other biologically inert fluid, is used to fill the balloon 22. Balloon 24 is inflated and deflated by a similar or different mechanism. In one embodiment of the present invention the same inflation mechanism is used for both balloons 22 and 24. In an alternative embodiment, each balloon is inflated by an independent mechanism, lumen and aperture. Such an embodiment may be useful where separately controlled inflation is desired. Other occluding mechanisms known to those skilled in the art other than balloons that can be increased or decreased in diameter are contemplated as being within the scope of the present invention.

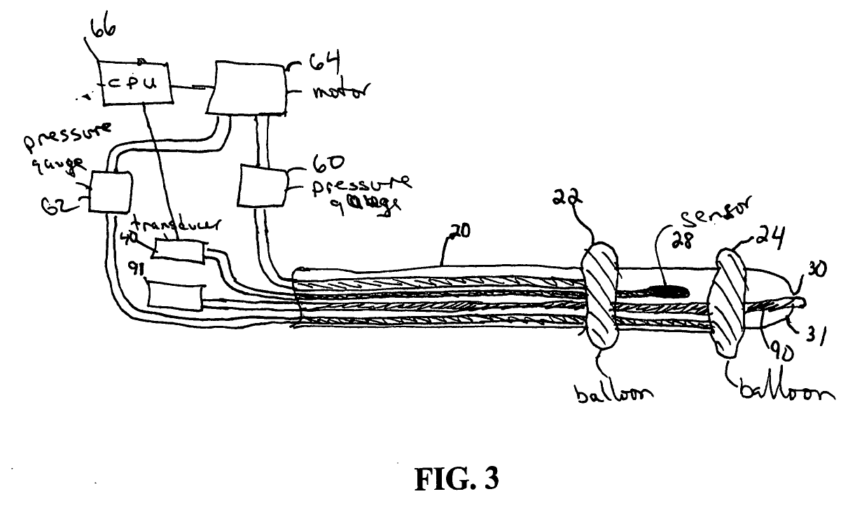

In a preferred embodiment the sensor 28 is a pressure sensor which communicates via a wire 38 (see FIGS. 3-5) or other communication conduit to an external pressure transducer 40, which is connected to a monitoring device 42, which can be a monitor, computer, electronic or display readout, or other device known to those skilled in the art as described in greater detail below. In an alternative embodiment the sensor 28 can be adapted to measure partial oxygen pressure, oxygen saturation, concentration of other fluid or gas components, particulate matter or the like.

FIG. 3 shows one embodiment of an automatic feedback loop circuit for an automatic inflation/deflation/measurement system using the apparatus 10 of the present invention. The balloons 22 and 24 are connected by tubing to individual pressure gauges 60 and 62 (or, alternatively, to a single pressure gauge). The pressure gauges 60 and 62 measure inflation pressure of the balloons 22 and 24 to ensure proper inflation. The gauges 60 and 62 are connected to at least one motor 64 which is capable of inflating or deflating the balloons by injecting or removing a fluid, such as saline as described above, in response to actuation. Alternatively, an inflation motor and a separate deflation motor can be employed for each balloon if separate functionality is desired. Alternatively, the balloons 22 and 24 can be inflated by a manually operated pump, syringe or other inflating device known to those skilled in the art. The motor 64 in turn is connected to a computer CPU (central processing unit) 66. The CPU 66 is also in communication with the sensor 28 by being connected to the sensor 28 by a fiber optic filament, wire, wireless or other connection known to those skilled in the art or developed hereafter.

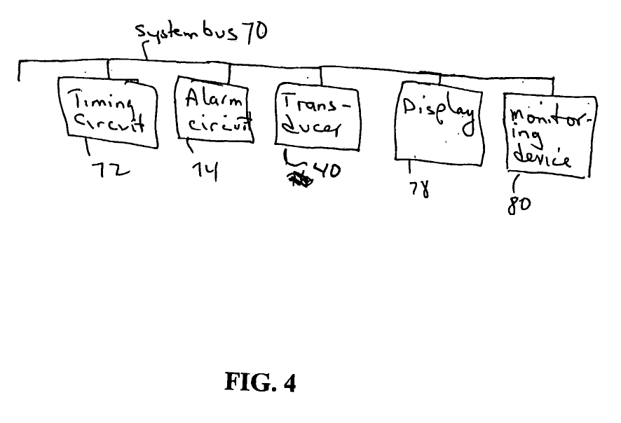

FIG. 4 shows a schematic diagram of the details of the CPU 66. A system bus 70 connects a timing circuit 72, an alarm circuit 74, a transducer measurement circuit 40, a display device 78, a connection to an external monitoring device 80 (such as, but not limited to, blood pressure cuff, EKG circuit, or the like), and/or other component. The timing circuit 72 can periodically (e.g., every five minutes) cause the CPU 66 to actuate the motor 64, which can automatically inflate the balloons 22 and 24. Upon full inflation the pressure gauges 60 and 62 would indicate proper inflation has been achieved and send a signal to the CPU 66 to deactuate the motor 64 and maintain inflation. Stable balloon inflation pressure can then be maintained. The sensor 28 can transmit sensor information to the transducer circuit 76 for a given period of time. Upon completion of pressure or other measurement or procedure, the timing circuit 72 instructs the CPU 66 to actuate the motor 64 to deflate the balloons 22 and 24 by withdrawing fluid which then collapses the balloons 22 and 24. It is also possible to partially inflate the balloons 22 and 24 by adjusting the feedback pressure measurement system of the motor 64 or other inflation/deflation device.

Measurements can be stored, analyzed and reported by the CPU 66 and displayed on display 78, which can be a CRT, LCD or other monitor, screen, tape strip, readout or printout or the like. If balloon pressure is not returned to nominal within a certain period of time post-measurement, as measured by the pressure gauges 60 and 62, an alarm circuit 74 will detect the nondeflation and actuate an alarm to warn the practitioner of possible malfunction and undesired blood vessel occlusion. The alarm 74 can be audible, visual or electronic signal to a remote location, such as a nurse's station, pager, cell phone, handheld computer or the like. The monitoring device 80 can be an external monitor which can confirm circulatory blood flow or occlusion, such as by measuring blood pressure, EKG, central venous pressure (such as by a catheter inserted into the right atrium) or other measurement device. This can minimize the possibility of detrimental occlusion by a faulty non-collapsing of a balloon by setting off an alarm if circulatory flow is not timely reestablished. In this manner a feedback loop system with internal and external failsafe mechanisms is provided to automatically take continuous period pressure measurements while minimizing prolonged disruption to blood flow from the brain.

In an alternative embodiment, an imaging device 90 can be included in an additional lumen in the catheter 20 for imaging the blood vessel wall or cell 50 contents. The imaging device can operatively be connected with a detection and/or processing system 91 for viewing or measurement.

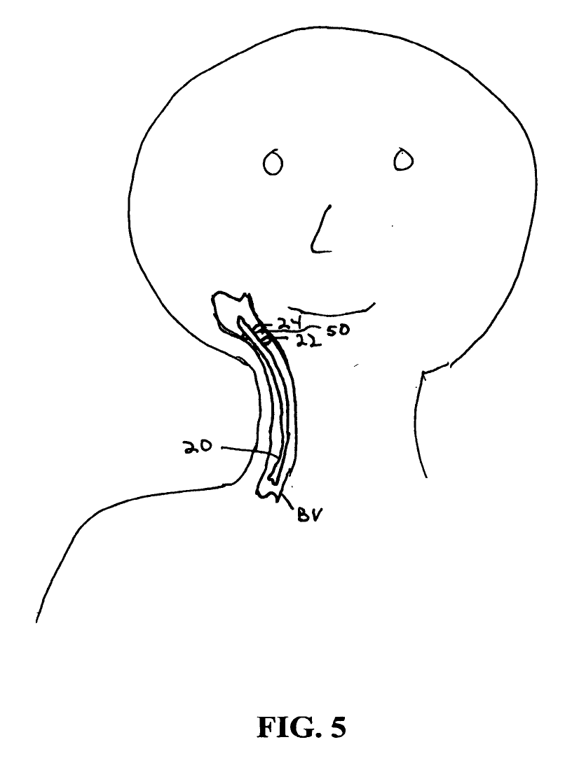

In operation (as shown in FIG. 5), the catheter 20 is introduced into a blood vessel, such as a vein, by venous puncture methods such as a Seldinger procedure known to those skilled in the art. The catheter 20 is introduced by percutaneous puncture into a vein, such as one in the neck, arm or groin (for a transfemoral insertion procedure), and advanced to the internal jugular vein at the base of the cranial cavity, just intracranial and downstream from where the jugular vein attaches to the skull. This portion of the jugular vein, known as the jugular bulb, is a key point of positioning (where the present invention is to be used for ICP measurement) because the vein in this region is compliant and thus capable of transducing pressure. Further up toward the brain the jugular vein is attached to the skull and has increased rigidity and diminished collapsibility. Correct positioning of the catheter 20 can be determined by angiography or other technique. For an ICP measurement procedure, once the catheter 20 is in position the motor 64 or other inflation device is actuated causing inflation of the proximal balloon 22. The distal balloon 24 is then inflated and deployed to contact the interior blood vessel wall so as to occlude blood flow. Proper inflation is measured by the pressure differential. When both balloons 22 and 24 are deployed, the motor 64 is deactuated and substantially constant pressure is maintained within the balloons 22 and 24. In this manner a discrete cell 50 is created by the interior blood vessel wall and the two balloons 22 and 24, as shown in FIG. 2 (blood flow direction being indicated by arrow 31). The sensor 28 can then take a reading of a stable fixed cell environment to determine pressure, component concentration, markers of brain injury (e.g., products of brain metabolism) or the like.

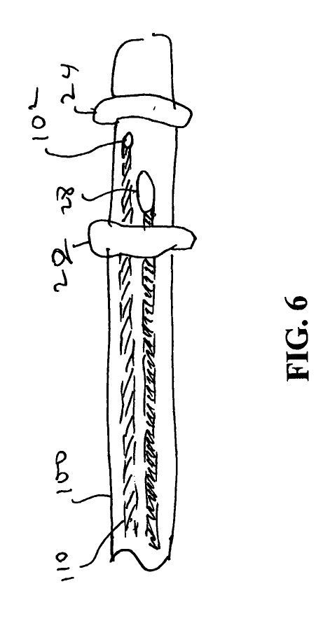

For a procedure in which a therapeutic or other substance is to be delivered to the site, it is possible for the distal balloon 24 to be inflated first, then the proximal balloon 22, followed by introduction of the therapeutic or other material. This embodiment can be used in brain oxygenation procedures to measure the therapeutic effect of delivery of an agent into the brain. For example, and not by way of limitation, mannitol can be delivered to the brain and its effects on ICP can be measured by an intracranial sensor. Alternatively, both balloons 22 and 24 can be inflated simultaneously. In an alternative embodiment shown in FIG. 6, a catheter 100 can have an additional aperture 102 associated with an additional lumen 110 for infusion or removal of fluid or other material within the cell 50 vicinity.

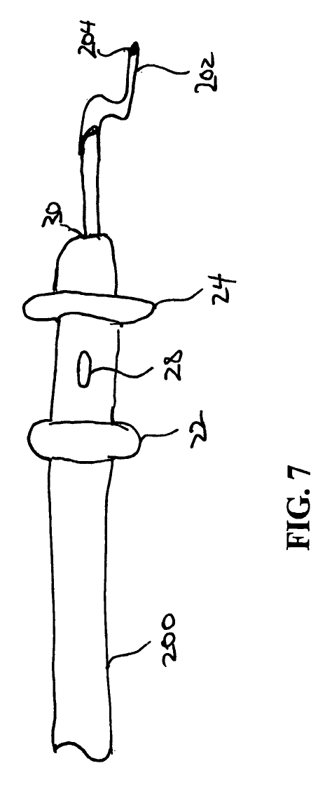

In a further alternative embodiment, illustrated in FIG. 7 a catheter 200 has a flexible probe 202 extendable through the aperture 30. The probe 202 can incorporate an emitter 204 at the distal tip of the probe 202 capable of extending within the vein beyond the jugular bulb 206 into the skull and emitting a detectable signal (e.g., light, infrared, ultraviolet, laser, microwave, ultrasound, other electromagnetic radiation, or the like). At least one, and preferably a plurality of external detectors 210 can be positioned, e.g., extracranially, to detect the signal emitted by the emitter 204.

In a further alternative embodiment, the probe 202 discussed above can be adapted to be a combination of a detector and an emitter. In such an embodiment the detector can emit light or other radiation which is detected by extracranial sensors. Such an embodiment can be used to detect and measure cerebral tissue oxygen concentration or presence of hematoma. A variation on this alternative embodiment is for the use of a single fiber or bundle of fibers which is operatively connected to an external detector and emitter. The same fiber can emit light or radiation and also be a detector. The emission and detection can be alternately pulsed.

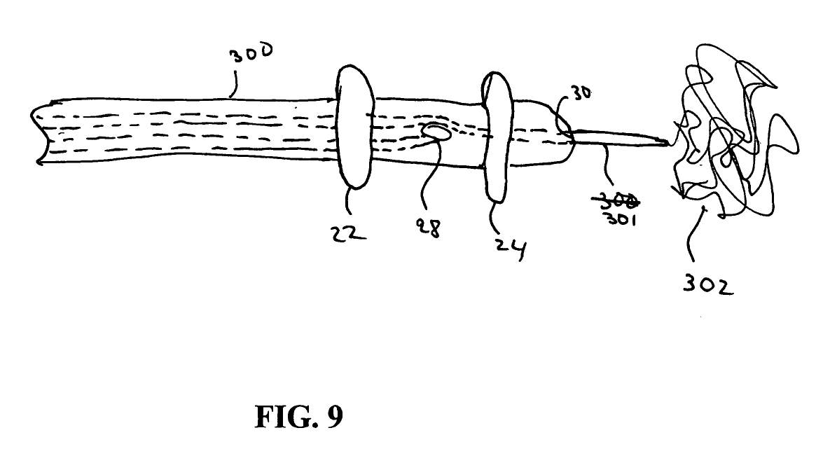

In another alternative embodiment, shown in FIG. 9, a catheter 300 has an aperture 30 which can accommodate a lumen 301 passing therethrough capable of delivering an optical or other type of dye 302. The dye 302, such as, but not limited to, indocyanine green or the like, can be injected upstream from the catheter 20 and dye concentration dilution can be measured downstream by the sensor 28. In such an embodiment the balloons 22 and 24 can be in a deflated condition.

A kit according to the present invention includes: a catheter 20 system as described above (in the preferred or alternative embodiments) where the catheter can be one of at least two different lengths depending on the insertion point, e.g., for transfemoral insertion, the catheter may be about 125 cm, and for jugular insertion can be about 30 cm, it being understood that the actual length is not critical so long as the practitioner can introduce the catheter 20 at the desired insertion point and reach the target position; a syringe with a thin walled introducer needle; and, an exchange wire. The syringe and exchange wire are known to those skilled in the art and commercially available or adaptable for the present invention. Optionally, a thin flexible guidewire can be included for navigating in the region of the jugular bulb. This thin guidewire may be preferably used where the sensor 200 is passed upstream from the jugular bulb. The kit may also contain an introducer sheath; a plastic sheath; an anesthetic; a topical antiseptic; a device to make an incision; sterile gauze; a dilator; and, a syringe to draw and deliver said anesthetic. Other components known to those skilled in the art can also be incorporated. Alternatively, a selection of several of the same components (e.g., needles) but of varying sizes can be included for convenience.

In a preferred embodiment of the kit of the present invention the kit is designed for use in insertion at a specific area. Thus, a transfemoral insertion kit would include a longer catheter 20 whereas a jugular insertion kit would contain a shorter catheter 20. Also, the needle or other component may differ in size, shape or length.

The present invention provides a method of measuring intracranial pressure (ICP), comprising the steps of:

a. providing a catheter comprising:

i) a catheter housing comprising a generally cylindrical tube having

(1) a sidewall,

(2) a proximal portion and

(3) a distal portion, said distal portion having at least one port defined at the end thereof,

ii) a first lumen at least partially and axially disposed within said catheter housing,

iii) a first expandable member capable of expanding from an initial volume to a deployed volume, said deployed volume being greater in size in at least one plane than said initial volume, said first expandable member being at least partially attached to said catheter housing,

iv) a first aperture defined in said catheter housing whereby said first lumen and said first expandable member are in fluid communication with each other through said first aperture,

v) a second lumen at least partially and axially disposed within said catheter housing,

vi) a second expandable member capable of expanding from an initial volume to a deployed volume, said deployed volume being greater in size in at least one plane than said initial volume, said second expandable member being at least partially attached to said catheter housing,

vii) a second aperture defined in said catheter housing whereby said second lumen and said second expandable member are in fluid communication with each other through said second aperture,

viii) a sensor disposed at least partially within said catheter housing said sensor having a distal end and a proximal end, said distal end extending through said catheter side wall and being positioned between said first expandable member and said second expandable member,

ix) a detector in communication with said sensor,

b. introducing said catheter into a blood vessel of a patient such that said distal portion of said catheter housing is moved into the vicinity of the jugular bulb,

c. deploying one of said expandable members;

d. deploying the other of said expandable members such that substantially all fluid flow within said blood vessel between said deployed expandable members has been occluded and a cell has been created by said expandable members and the wall of said blood vessel;

e. sensing the environment within said cell by said sensor; and,

f. measuring the fluid pressure in said cell.

Advantages

A significant advantage of the present invention is that invasive measurement of ICP in the subarachnoid, intraventricular, intraparenchymal or epidural compartments requires craniotomy, whereas this technique does not. Non-neurosurgery trained personnel can use the present invention, thus lowering cost and time involved. The present invention may reduce risk of further brain injury and of trauma to the cranial vicinity by providing extracranial, endovascular access to intracranial pressure measurement. The catheter of the present invention can remain indwelling in a patient for days with minimal risk of causing injury or deleterious effects on the patient. Rapid recalibration of a unit or replacement of a defective or malfunctioning unit can be achieved with minimal trauma to the patient. The catheter of the present invention also offers the opportunity to compare products draining into the cerebral venous blood to those in the peripheral venous circulation. This systematic sampling of cerebral and peripheral blood can be used to detect substances being produced exclusively in the brain, which are unmeasurable when diluted with the rest of the circulation volume (approximately 5 liters). In addition the present invention can be used to assess the efficacy of drugs or biologics which are designed to alter the production of certain substances or inhibit certain chemical or biological reactions.

The present invention can be adapted for use in a commercial or industrial setting where continuous pressure measurement in a tube is needed. For many such applications pressure measurement may not require collapsibility of the tube wall, thus, rigid or flexible tubing (in place of the blood vessel) can be used.

The invention will be further described in connection with the following example, which is set forth for purposes of illustration only.

### EXAMPLE

### Example 1

A catheter containing a pair of balloons and a sensor was introduced by venipuncture in a patient's neck into the jugular vein using conventional Seldinger procedure. The catheter was advanced intravenously into the jugular bulb area. Fiber optic sensor measurements were taken over time.

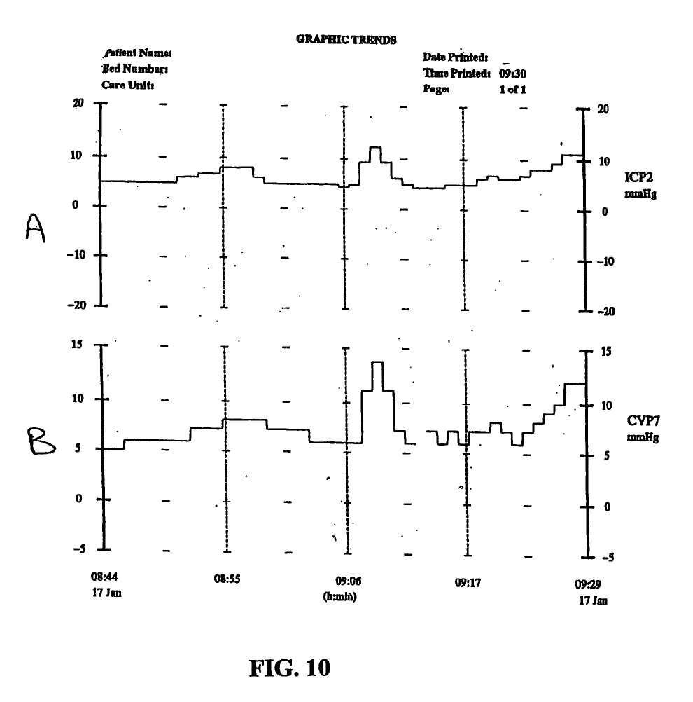

FIG. 10A is a graph showing trends over time of conventionally measured ICP measurement via ventriculostomy compared with measurement via the jugular bulb catheter (labeled CVP, cerebral venous pressure) (See FIG. 10B) in a patient. Note that the scales are different. The correlation is excellent between the two measurements, indicating that in this patient jugular bulb pressure is an accurate reflection of ICP.

Although only a few exemplary embodiments of this invention have been described in detail above, those skilled in the art will readily appreciate that many modifications are possible in the exemplary embodiments without materially departing from the novel teachings and advantages of this invention.

It should further be noted that any patents, applications or publications referred to herein are incorporated by reference in their entirety.

Claimed is:

1. A catheter, comprising:
a. a catheter housing comprising a generally cylindrical tube having
i) a sidewall,
ii) a proximal portion and
iii) a distal portion, said distal portion having at least one port defined at the end thereof,
b. a first lumen at least partially and axially disposed within said catheter housing,
c. a first expandable member being at least partially attached to said catheter housing,
d. a first aperture defined in said catheter housing whereby said first lumen and said first expandable member are in fluid communication with each other through said first aperture,
e. a second lumen at least partially and axially disposed within said catheter housing,
f. a second expandable member being at least partially attached to said catheter housing,
g. a second aperture defined in said catheter housing whereby said second lumen and said second expandable member are in fluid communication with each other through said second aperture,
h. a sensor disposed at least partially within said catheter housing said sensor having a distal end and a proximal end, said distal end extending through said catheter side wall and being positioned between said first expandable member and said second expandable member, and
i. a detector in communication with said sensor.

2. The catheter of claim 1, wherein said first expandable member is capable of expanding from an initial volume to a deployed volume, said deployed volume being greater in size in at least one plane than said initial volume.

3. The catheter of claim 1, wherein said second expandable member is capable of expanding from an initial volume to a deployed volume, said deployed volume being greater in size in at least one plane than said initial volume.

4. A method of measuring intracranial pressure (ICP), comprising the steps of:
a. providing a catheter comprising:
i) a catheter housing comprising a generally cylindrical tube having
(1) a sidewall,
(2) a proximal portion and
(3) a distal portion, said distal portion having at least one port defined at the end thereof,
ii) a first lumen at least partially and axially disposed within said catheter housing,
iii) a first expandable member capable of expanding from an initial volume to a deployed volume, said deployed volume being greater in size in at least one plane than said initial volume, said first expandable member being at least partially attached to said catheter housing,
iv) a first aperture defined in said catheter housing whereby said first lumen and said first expandable member are in fluid communication with each other through said first aperture,
v) a second lumen at least partially and axially disposed within said catheter housing,
vi) a second expandable member capable of expanding from an initial volume to a deployed volume, said deployed volume being greater in size in at least one plane than said initial volume, said second expandable member being at least partially attached to said catheter housing,
vii) a second aperture defined in said catheter housing whereby said second lumen and said second expandable member are in fluid communication with each other through said second aperture,
viii) a sensor disposed at least partially within said catheter housing said sensor having a distal end and a proximal end, said distal end extending through said catheter side wall and being positioned between said first expandable member and said second expandable member, and
ix) a detector in communication with said sensor;
b. introducing said catheter into a blood vessel of a patient such that said distal portion of said catheter housing is moved into the vicinity of the jugular bulb;
c. deploying one of said expandable members;
d. deploying-the other of said expandable members such that substantially all fluid flow within said blood vessel between said deployed expandable members has been occluded and a cell has been created by said expandable members and the wall of said blood vessel;
e. sensing the environment within said cell by said sensor; and,
f. measuring the fluid pressure in said cell.

5. A kit, comprising:
a. a catheter comprising:
i) a catheter housing comprising a generally cylindrical tube having
(1) a sidewall,
(2) a proximal portion and
(3) a distal portion, said distal portion having at least one port defined at the end thereof,
ii) a first lumen at least partially and axially disposed within said catheter housing,
iii) a first expandable member capable of expanding from an initial volume to a deployed volume, said deployed volume being greater in size in at least one plane than said initial volume, said first expandable member being at least partially attached to said catheter housing,
iv) a first aperture defined in said catheter housing whereby said first lumen and said first expandable member are in fluid communication with each other through said first aperture,
v) a second lumen at least partially and axially disposed within said catheter housing,
vi) a second expandable member capable of expanding from an initial volume to a deployed volume, said deployed volume being greater in size in at least one plane than said initial volume, said second expandable member being at least partially attached to said catheter housing,
vii) a second aperture defined in said catheter housing whereby said second lumen and said second expandable member are in fluid communication with each other through said second aperture,
viii) a sensor disposed at least partially within said catheter housing said sensor having a distal end and a proximal end, said distal end extending through said catheter side wall and being positioned between said first expandable member and said second expandable member, and
ix) a detector in communication with said sensor;
b. a syringe having an introducer needle associated therewith;
c. an introducer sheath;
d. a plastic sheath;
e. an anesthetic;
f. a topical antiseptic;
g. a device to make an incision;
h. sterile gauze;
i. a dilator;
j. a syringe to draw and deliver said anesthetic; and,
k. an exchange wire.
